(19) United States
(12) Patent Application Publication
(10) Pub. No.: US 2023/0218180 A1
(43) Pub. Date: Jul. 13, 2023

(54) MONITORING SYSTEMS AND DEVICES FOR HEART IMPLANTS

(71) Applicant: EDWARDS LIFESCIENCES CORPORATION, Irvine, CA (US)

(72) Inventors: Muhammad Mujeeb-U-Rahman, Irvine, CA (US); Alexander H. Siemons, Yorba Linda, CA (US); Rendle Lamarr Myles, JR., Covina, CA (US)

(21) Appl. No.: 18/174,332

(22) Filed: Feb. 24, 2023

Related U.S. Application Data
(63) Continuation of application No. PCT/US21/46375, filed on Aug. 17, 2021.
(60) Provisional application No. 63/072,298, filed on Aug. 31, 2020.

(51) Int. Cl. A61B 5/0215; A61B 5/00; A61F 2/24

(52) U.S. Cl. CPC A61B 5/02158; A61B 5/686; A61B 5/4851; A61B 5/0031; A61F 2/2418; A61B 2562/04; A61B 2560/0219; A61B 2562/164; A61F 2250/0001

(57) ABSTRACT

A prosthetic valve comprises a frame assembly having a first opening at an inflow portion of the frame assembly and a second opening at an outflow portion of the frame assembly, a first sensor device situated at the inflow portion of the frame, and a second sensor device situated at the outflow portion of the frame. Each of the first sensor device and the second sensor device is configured to sense a physical parameter and provide a sensor signal. The prosthetic valve further comprises a transmitter assembly configured to receive the sensor signals from the first sensor device and the second sensor device and wirelessly transmit a transmission signal based at least in part on the sensor signals.

### RELATED APPLICATION

This application is a CON of International Patent Application No. PCT/US2021/046375, filed Aug. 17, 2021, which claims priority to U.S. Provisional Application No. 63/072,298, filed on Aug. 31, 2020, entitled MONITORING SYSTEMS AND DEVICES FOR HEART IMPLANTS. Each of the foregoing patent applications are hereby incorporated by reference in their entireties.

### BACKGROUND

The present disclosure generally relates to the field of medical implant devices.

Various medical procedures involve the implantation of medical implant devices within the anatomy of the heart. Certain physiological parameters associated with such anatomy, such as fluid pressure, can have an impact on patient health prospects.

### SUMMARY

Described herein are one or more methods and/or devices to facilitate monitoring of physiological parameter(s) associated with the left atrium using one or more sensor implant devices implanted in or to one or more pulmonary veins and/or associated anatomy/tissue.

Some implementations of the present disclosure involve a prosthetic valve comprising a frame assembly having a first opening at an inflow portion of the frame assembly and a second opening at an outflow portion of the frame assembly, a first sensor device situated at the inflow portion of the frame assembly, and a second sensor device situated at the outflow portion of the frame assembly. Each of the first sensor device and the second sensor device is configured to sense a physical parameter and provide a sensor signal. The prosthetic valve further comprises a transmitter assembly configured to receive the sensor signals from the first sensor device and the second sensor device and wirelessly transmit a transmission signal based at least in part on the sensor signals.

In some embodiments, the frame assembly is configured to support a first post extending from the inflow portion of the frame assembly and a second post extending from the outflow portion of the frame assembly. The first sensor device may be situated at the first post and the second sensor device may be situated at the second post. In some embodiments, the first sensor device is configured to slide within the first post.

The prosthetic valve may further comprise a third sensor device at the inflow portion of the frame assembly. In some embodiments, the prosthetic valve further comprises a substrate band. The first sensor device and the third sensor device may be coupled to the substrate band.

In some embodiments, the prosthetic valve further comprises a first substrate extension extending axially from the substrate band to the outflow portion of the frame assembly. The second sensor device may be coupled to the substrate extension.

The first substrate extension may have a non-linear structure. In some embodiments, the prosthetic valve further comprises a first substrate extension extending diagonally from the substrate band to the outflow portion of the frame assembly.

In some embodiments, the first sensor device and the second sensor device are composed of a polymer material.

The transmitter assembly may include an electrically conductive coil configured to wirelessly transmit the transmission signal.

In some embodiments, the first sensor device is powered via wireless powering.

Some implementations of the present disclosure relate to a patient monitoring system comprising a prosthetic valve implant device configured to be implanted in a patient. The prosthetic valve implant device includes a frame assembly configured to support a first post extending from an inflow portion of the frame assembly and a second post extending from an outflow portion of the frame assembly, a first sensor device situated at the first post, a second sensor device situated at the second post, wherein each of the first sensor device and the second sensor device is configured to sense a physical parameter and provide a sensor signal, and a wireless transmitter assembly configured to receive the sensor signals from the first sensor device and the second sensor device and wirelessly transmit a transmission signal based at least in part on the sensor signals. The patient monitoring system further comprises a receiver device configured to wirelessly couple with the wireless transmitter assembly of the prosthetic valve implant device and receive the transmission signal while the prosthetic valve implant device is implanted in a patient and the receiver devices is located external to the patient.

The patient monitoring system may further comprise a third sensor device at the inflow portion of the frame assembly. In some embodiments, the patient monitoring system further comprises a substrate band, wherein the first sensor device and the third sensor device are coupled to the substrate band.

The first sensor device and the second sensor device may be composed of a polymer material.

Some implementations of the present disclosure relate to a method of monitoring a prosthetic implant patient. The method comprises wirelessly coupling an external receiver device to a prosthetic valve implant device implanted in a patient, measuring a physical parameter associated with the patient using a sensor device of the prosthetic valve implant device, and wirelessly transmitting a signal based on the measurement of the physical parameter using a transmitter assembly. The transmitter assembly includes a frame assembly having a first opening at an inflow portion of the frame assembly and a second opening at an outflow portion of the frame assembly, a first sensor device situated at the inflow portion of the frame assembly, a second sensor device situated at the outflow portion of the frame assembly, wherein each of the first sensor device and the second sensor device is configured to sense a physical parameter and provide a sensor signal, and a transmitter configured to receive the sensor signals from the first sensor device and the second sensor device and wirelessly transmit a transmission signal based at least in part on the sensor signals.

In some embodiments, the frame assembly is configured to support a first post extending from the inflow portion of the frame assembly and a second post extending from the outflow portion of the frame assembly.

The first sensor device may be situated at the first post and the second sensor device may be situated at the second post.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a schematic drawing of a human heart.

FIG. 2 provides a schematic drawing of a surgical prosthetic heart valve implanted in a heart according to one or more embodiments.

FIG. 3 is a block diagram representing an implant device in accordance with one or more embodiments.

FIG. 4 is a block diagram representing a system for monitoring one or more physiological parameters associated with a patient according to one or more embodiments.

FIG. 5 provides a schematic drawing of an example circuit for one or more sensors as described herein which may be attached to an artificial valve for gathering and/or wirelessly transmitting data to an external receiver in accordance with one or more embodiments.

FIG. 6 depicts an example frame comprising a network of struts forming one or more cells in accordance with one or more embodiments.

FIG. 7 illustrates a prosthetic valve including a frame and one or more posts extending from the frame in accordance with one or more embodiments.

FIG. 8 illustrates a prosthetic valve comprising a frame and one or more sensors at one or more posts extending from the frame in accordance with one or more embodiments.

FIG. 9 illustrates another valve comprising a frame and a substrate band wrapped at least partially about a circumference at or near a first portion of the frame in accordance with one or more embodiments.

FIG. 10 illustrates a valve comprising a frame and a skirt wrapped at least partially around an inner surface and/or outer surface of the frame in accordance with one or more embodiments.

FIG. 11 illustrates how a valve alignment can be altered as a result of breathing and/or other chest movement of a patient.

FIG. 12 illustrates a frame comprising a substrate band and one or more substrate extensions having an extendible structure in accordance with one or more embodiments.

FIG. 13 illustrates another example valve including a frame, a substrate band, and one or more substrate extensions configured to extend from the substrate band at a first portion of the frame to a second portion of the frame in accordance with one or more embodiments.

FIG. 14 illustrates a valve including a frame and one or more posts configured to allow one or more sensors to slide within the posts to adjust the positions of the one or more sensors with respect to the posts and/or frame in accordance with one or more embodiments.

FIG. 15 is a flow diagram illustrating a process for monitoring a postoperative implant device and/or patient associated therewith in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for telemetric monitoring of one or more physical/physiological parameters of a patient (e.g., blood pressure) in connection with cardiac shunts and/or other medical implant devices (e.g., prosthetic valve implant devices) and/or procedures. Such pressure monitoring may be performed using cardiac implant devices (e.g., prosthetic valve implant devices) having integrated pressure sensors and/or associated components. For example, in some implementations, the present disclosure relates to cardiac shunts and/or other cardiac implant devices that incorporate or are associated with pressure sensors or other sensor devices. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly. Certain embodiments are disclosed herein in the context of cardiac implant devices. However, although certain principles disclosed herein are particularly applicable to the anatomy of the heart, it should be understood that sensor implant devices in accordance with the present disclosure may be implanted in, or configured for implantation in, any suitable or desirable anatomy. The placement of an artificial valve or stent inside the heart of a patient can provide a unique opportunity to measure cardiac function as well. This can have clinical applications in monitoring cardiovascular health without requiring separate implants. The present solution provides a system for implanting cardiac implant devices including certain sensor and wireless transmission components and collecting data therefrom using a handheld reading device containing a suitable RF antenna to read a transmission signal from the implant device (e.g., prosthetic heart valve). Such systems can be used to monitor patients during and/or after valve implantation to validate proper operation using monitoring rather than spot checks using bio-imaging. These solutions provide options for monitoring valve status in real-time and/or for a large set of patients over relatively long postoperative periods.

Some embodiments may be configured to operate via wireless powering and/or wireless communication and/or may consist of several components including a heart valve with one or more integrated sensors, an external readout unit consisting of a matching antenna, a signal processing unit (e.g., configured to transmit and/or receive transmission signals), and wireless link to a secure cloud and a patient monitoring system. Some systems can include soft and/or bio-compatible sensors that can be used with existing medical implants (e.g., prosthetic valves) and delivery systems. Some embodiments may provide soft-sensing platforms that can be developed using standard soft and bio-compatible materials that can be wrapped at least partially around a valve assembly without substantially affecting blood flow, which may allow for safe and effective application over extended periods. In contrast, hard-anchored sensors may be unable and/or difficult to crimp, can be difficult to integrate with valves, and/or can cause significant thrombus during operation.

Wireless communication may be facilitated using, for example, an inductor-capacitor (LC) resonant structure including one or more coil inductors and/or thin-film membrane-based capacitors. An LC resonance frequency may be adjusted to match with an external source configured to energize the system by sending electromagnetic excitations at the same frequency (i.e., at resonance frequency). The resonance frequency may be chosen to minimize loss in the tissue and/or to maximize energy transfer to the resonance coil, while avoiding minimum reflections and/or interference from the frame (e.g., at least partially metallic frame) of the valve. The terms “frame” and “frame assembly” are used herein according to their plain and ordinary meaning and may include any components forming a structure of an implant device (e.g., a prosthetic valve). In some embodiments, a frame or frame assembly may include a network of struts forming one or more cells around an internal lumen.

One or more LC sensors can comprise one or more flexible substrates, inductive coils, capacitive pressure sensors, chips to multiplex and/or transmit data wirelessly, and/or fixed capacitors. In some cases, LC sensor can be configured to simultaneously monitor multiple different parameters.

One or more wireless sensors can be implanted at different portions of an artificial valve. For example, a sensor can be contained fully within a valve body, comprise multiple separate sensing units situated at either end of the valve body, and/or comprise a main sensor unit with an ancillary unit attached to it. The sensor(s) can be crimped to smaller diameters to fit within a crimped valve. When the assembly is expanded, the sensor(s) can be pulled inside the valve using mechanical attachments. The sensor(s) and/or valve can be at least partially composed of metal but can have different structures to support their respective expansion during valve deployment.

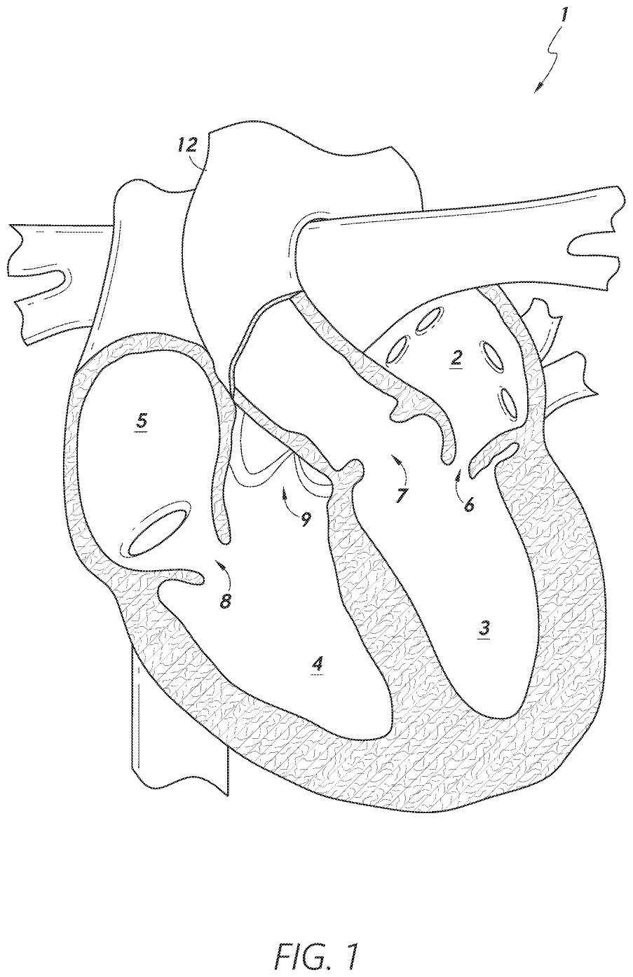

Embodiments of heart valve monitoring devices and systems disclosed herein may be applicable with respect to any type of heart valve and/or bio-compatible implant, whether implanted using surgical or transcatheter means. FIG. 1 provides a schematic drawing of a human heart 1. In humans and other vertebrate animals, the heart 1 generally includes four chambers, namely the left atrium 2, the left ventricle 3, the right ventricle 4, and the right atrium 5. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The pulmonary valve 9 separates the right ventricle 4 from the pulmonary artery and may be configured to open during systole so that blood may be pumped towards the lungs, and close during diastole to prevent blood from leaking back into the heart from the pulmonary artery. The pulmonary valve 9 has three cusps/leaflets, each one resembling a crescent. The mitral valve 6 has two cusps/leaflets and separates the left atrium 2 from the left ventricle 3. The mitral valve 6 is configured to open during diastole so that blood in the left atrium 2 can flow into the left ventricle 3, and close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Some valves may further comprise a collection of chordae tendineae and papillary muscles securing the leaflets. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated.

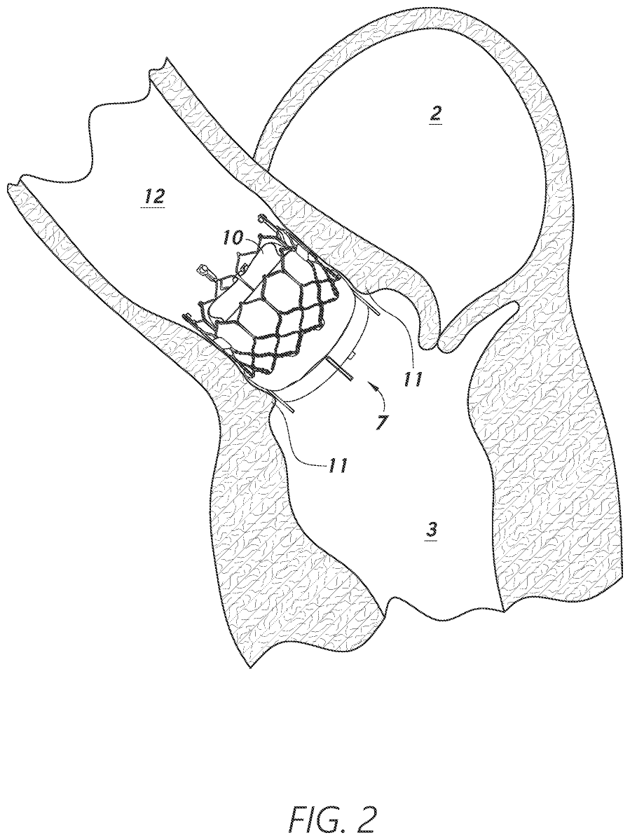

FIG. 2 provides a schematic drawing of a surgical prosthetic heart valve 10 implanted in a heart 1 according to one or more embodiments. In certain embodiments, the heart valve 10 may include one or more sensors (not shown) for measuring/sensing one or more physical/physiological parameters, as described herein. The heart valve 10 may further include means for wirelessly transmitting signals associated with the sensor response to an external receiver device, wherein such means may include a wireless transmitter or transceiver, for example.

The heart valve 10 may function to allow fluid flow in one direction, such as out of the heart with respect to an aortic heart valve, while inhibiting fluid flow in the opposite direction. The heart valve 10 represents an exemplary surgical prosthetic heart valve, which is shown implanted in the aortic valve 7. However, it should be understood that heart valves as disclosed herein may be any type of heart valve. FIG. 2 provides an enlarged view of the aortic valve 7 shown in FIG. 1. The aortic valve 7 includes an aortic annulus 11, which comprises a fibrous ring extending inward as a ledge into the flow orifice and can be seen with the prosthetic heart valve 10 disposed thereon (e.g., sutured thereto). Prior to valve replacement, the native leaflets may extend inward from the annulus 11 and come together in the flow orifice to permit flow in the outflow direction (e.g., the upward direction in FIG. 2) and prevent backflow or regurgitation toward the inflow direction (e.g., the downward direction in FIG. 2).

In a typical cardiac implant procedure, the aorta may be incised and, in a valve replacement operation, the defective valve may be removed leaving the desired placement site that may include the valve annulus. Sutures may be passed through fibrous tissue of the annulus or desired placement site to form an array of sutures. Free ends of the sutures may be individually threaded through a suture-permeable sealing edge of the prosthetic heart valve.

Artificial heart valves can be used to replace faulty or deteriorating natural heart valves in patients with heart valve disorders including aortic stenosis, mitral regurgitation, etc. The valve replacement process generally involves surgical or transcatheter procedures to replace the existing valves with the new artificial valves. Since the artificial valves are a foreign body, many different challenges and issues can be involved with such a procedure. For example, paravalvular leakage (PVL) occurs in around 10% of patients who undergo Transcatheter Aortic Valve Replacement (TAVR). Leaflet thickening is another issue that occurs in around 10% of TAVR patients. Similarly, rejection of an artificial surgical heart valve due to thrombus can occur, requiring the patient to use anti-coagulants for proper valve operation.

Some methods for monitoring valve performance after implantation involve using complex bio-imaging techniques, such as echocardiography. Such methods can generally only be performed in specialized medical facilities and can cost significant time and money. Hence, such methods may generally only be used once symptoms of valve malfunction are detected. Some artificial valves may not provide an ability to detect changes in operation to detect problems early on. Moreover, many patients who suffer from valvular disease and require an artificial valve may also suffer from other cardiovascular disorders, including heart failure. Some artificial heart valve systems may not allow for gathering data about the valve and/or the patient's condition postoperatively in an outpatient setting (e.g., a cardiologist visit in a ward) using existing patient monitoring systems. Such systems may not provide for routine collection of data at sufficient resolution to enable development of new digital solutions for better management of the patients as their numbers and diversity increase over time.

Various surgical techniques may be used to replace or repair a diseased or damaged valve, including securing a cardiac implant to the diseased annulus. Cardiac implants include mechanical prosthetic heart valves, valved conduits and annuloplasty rings. In a valve replacement operation, damaged leaflets may be excised and the annulus sculpted to receive a replacement valve.

Prosthetic heart valves may be composed of various synthetic and/or biologically-derived materials/tissues. Prosthetic heart valves may be implanted independently in one of the orifices and/or annuluses of the heart, and/or may be otherwise coupled to a flow conduit which extends in line with the valve. For example, valved conduits can be designed for reconstruction of portions of the flow passage above and below the aortic valve, such as the ascending aorta, in addition to replacing the function of the valve itself. Introduction of the sensors into the patient system may be through surgical or minimally-invasive means.

Patients who receive heart valve implants may suffer from post-operation complications. For example, a patient may be particularly susceptible to complications within thirty or sixty days of an implant operation. However, during such periods of time, the patient may no longer be in a hospital or extended care facility/system, and therefore complications that arise may require reentry into the care system, potentially adding significant cost to the overall patient treatment. Furthermore, increased health risks may result from the patient delaying return to the hospital due to failure to recognize the complications until they manifest through perceivable symptoms that the patient interprets as requiring hospital care.

Disclosed herein are systems, devices and methods for post-operatively monitoring prosthetic heart valve implant recipients, including possibly in an environment outside of the hospital or care facility. Certain embodiments disclosed herein provide a heart valve device/system including integral sensing capability for sensing one or more conditions of the heart valve and/or heart of a patient. The heart valve may be configured to wirelessly communicate such sensed parameters (e.g., critical patient issues) from the sensor system in the valve to a local or remote wireless receiver device, which may be carried by the patient in some embodiments. The receiver may be configured to communicate information associated with the received sensor information to a care provider system, such as to a remote hospital or care facility monitoring system. Sensor-integrated implant devices in accordance with principles disclosed herein may include surgical valves (e.g., aortic or mitral), transcatheter heart valves (THV), annuloplasty rings (e.g., mitral, tricuspid), pacemakers (e.g., in connection with electrical leads), or the like, or may alternatively be applicable to stand-alone sensor devices that are not integrated with a valve or other implant device.

Physiological parameters that may be tracked by sensor-enabled heart valve implants may include arrhythmia, blood pressure, cardiac output (e.g., as measured by an echo sensor, induction, ballistocardiogram, or the like), and/or other parameter(s). Furthermore, implant devices disclosed herein may incorporate any desired or practical types of sensors, such as strain gauges, pressure sensors, optical sensors, audio sensor, position sensors, or other type(s) of sensor. Integrated implant sensors may advantageously be configured to generate electrical transmission signals that may be wirelessly transmitted to a receiver device (e.g., box) disposed outside the patient's body. In certain embodiments, the receiver device may forward information based at least in part on the signals to a remote care giver system/entity.

In certain embodiments, sensor devices associated with implant devices may be configured to sense pressure and/or electrical activity. For example, pressure may provide information regarding how well the implant is functioning, as well as possibly information regarding hydration. Electrical activity sensor(s) may provide information used to detect arrhythmia. Pressure sensors integrated in devices in accordance with the present disclosure may include microelectromechanical systems (MEMS) devices (e.g., accelerometer), which may be integrated in the implant frame, for example. In certain embodiments, two or more sensors may be utilized. As an example, a plurality of sensors may be used to measure differential pressure between the inflow and outflow ends of a valve implant, which may provide information indicating regurgitation.

Sensors and/or transmitters integrated in implant devices according to embodiments of the present disclosure may only need to operate for a limited monitoring period of time (e.g., 90 to 120 days), and may therefore be powerable using a battery, such as a lithium ion or magnesium-based battery. For example, a battery may use a piece of magnesium as a cathode in at least partial contact with body fluid(s) (e.g., blood), which may degrade as it generates electrical power. In certain embodiments, an external power source configured to provide power through induction, radio frequency (RF) transmission, or other type of wireless power transmission may be used. In certain embodiments, an internal rechargeable battery or capacitor (e.g., supercapacitor) may be used for limited power storage between charging. Such a power transmitter may be integrated with an external data receiver. In certain embodiments, a portion of the frame of the implant/sensor device may be used as an antenna for power transmission. Additionally or alternatively, the patient's body movement may be used to generate power, such as by using one or more piezoelectric MEMS devices (e.g., strain gauge, accelerometer).

In certain embodiments, implant-integrated sensor devices may be configured to run substantially continuously. Alternatively, the sensor(s) may run only for predetermined intervals, which may provide power savings compared to continuous operation. In certain embodiments, controller logic may be integrated with the implant/sensor for determining timing and/or duration of operation based on measured conditions. In certain embodiments, the sensor(s) may operate only when wirelessly coupled with an external data/power transfer/receiving device. In embodiments in which the sensor(s) collect data even when the device is not coupled to an external device, it may be necessary or desirable for the implant/sensor to include data storage, such as flash memory, memristor(s), or other low-power memory.

Certain embodiments can operate in connection with an external power/data transfer device, which may advantageously be small enough to be carried with by the patient (e.g., continuously), such as by using a chest strap, or the like. In certain embodiments, the external device comprises a patch with one or more antennae for input/output (I/O) and/or power; remaining circuitry may be contained in a separate box/device. In certain embodiments, the external device may comprise an arm-strap fitted device, or a device that may fit in the patient's pocket. Bluetooth, near-field communication (NFC), or other low-power technology or protocol may be used to connect the external device and/or implant/sensor to a phone or other computing device to transmit data to a hospital or other data aggregator. In certain embodiments, the external device may comprise a mat designed to be located at or near a bed; the mat may collect data and transmit the data while the patient is sleeping, for example.

In some embodiments, data may be collected using the existing patient monitoring systems, which may include handheld reading devices containing a suitable radio frequency (RF) antenna to read transmission signals from an implanted valve. The received data can then be used to determine at-risk patients and/or prescribe various treatments, including use of anti-coagulants to prevent valve failure. Moreover, the data received from the implanted valve can be used to monitor patients during and right after valve implantation to validate proper operation.

The various device and systems described herein advantageously provide a monitoring system which can provide improved methods of monitoring valve status in real-time for a large set of patients over extended time periods. In some embodiments, a remote monitoring system can be used to monitor the state of an artificial heart valve and the condition and/or function of surrounding cardiac tissue. The remote monitoring system can operate via wireless powering and/or wireless communication and/or may comprise several components including a heart valve with one or more integrated sensors, an external readout unit comprising a matching antenna, a signal processing unit, and/or a wireless link to a secure cloud and/or a patient monitoring system.

In some embodiments, sensors described herein (e.g., soft and/or biocompatible sensors) can advantageously be used with existing valve and/or delivery systems and hence can require minimal efforts for development and validation. These soft-sensing platforms can be developed using standard soft and/or biocompatible materials that can be at least partially wrapped around the valve assembly and/or may not cause any significant effects on blood flow, which can be crucial for safe and effective applications over extended periods.

One or more integrated sensors can be maintained within the frame/body of the valve during normal operation. In some cases, it may be difficult to ensure reliable wireless power transfer due to the presence of the frame (e.g., a metal mesh) of the valve. Some embodiments may advantageously provide for one or more sensors to be situated outside of a central lumen of the frame of the valve structure to keep the sensing platform outside the valve to improve powering and/or communication with the remote sensing platform.

In some embodiments, one or more sensors and/or associated structure can be attached to a valve during manufacturing. One or more sensors may be configured to be held at least partially external to the valve using a mechanical post and latch structure. For example, for transcatheter procedures, one or more sensors may be pulled within the valve once the valve is deployed and expanded. In some embodiments, a relatively thin sensing platform may be used to allow the sensing platform to be held within the valve during and/or after valve implantation.

In some embodiments, the electrical design of the system may include an LC resonant structure comprising a coil inductor and/or a thin-film membrane-based capacitor. The LC resonance frequency may be adjusted to match with an external source that energizes the system by sending electromagnetic excitations at a common frequency (i.e., a resonance frequency). The frequency may be chosen to minimize loss in the tissue and/or to maximize energy transfer to the resonance coil while avoiding minimum reflections and/or interference from frame of the valve. In some embodiments, one or more resistor-inductor-capacitor (RLC) sensors, application specific integrated circuits (ASICs), radio-frequency identification (RFID) circuits, and/or near-field communication (NFC) circuits may be used with or in place of one or more LC sensors. The one or more sensors and/or surrounding structure may at least partially comprise biodegradable material that can absorb with time once the sensor lifetime is over. One or more sensors can at least partially comprise material that can actively encourage growth to enable controlled encapsulation around the valve to maintain a controlled environment for long-term measurement.

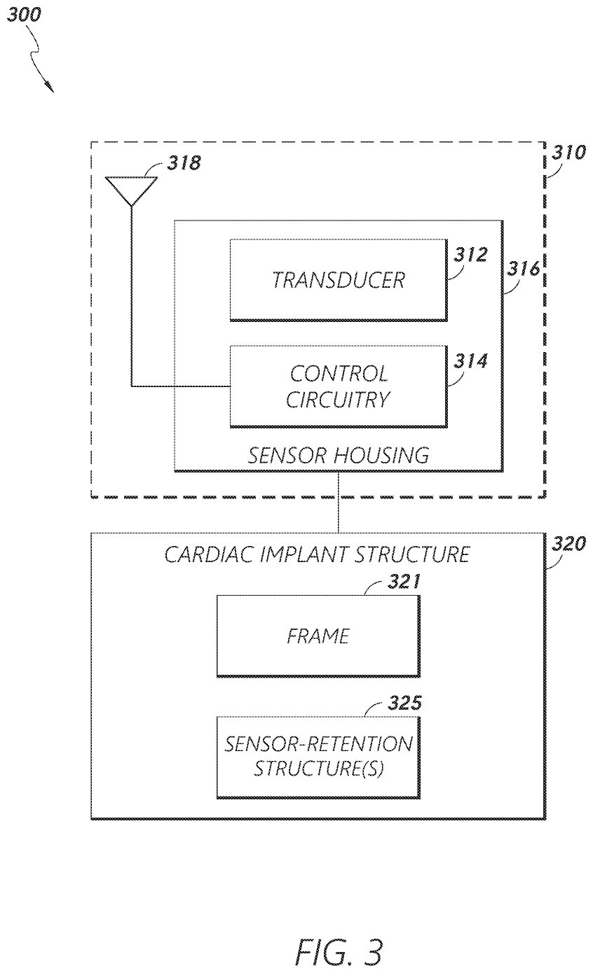

In some implementations, the present disclosure relates to sensors associated or integrated with cardiac shunts or other implant devices/structures. Such integrated devices may be used to provide controlled and/or more effective therapies for treating and preventing heart failure and/or other health complications related to cardiac function. FIG. 3 is a block diagram illustrating an implant device 300 comprising a cardiac implant structure 320, which may comprise a shunt-type structure, as described in detail herein, or any other type of implant structure. The cardiac implant structure 320 can include a frame 321 which may be configured to anchor the implant device 300 in place in the implant location/position. For example, the frame 321 may be configured to at least partially expand and/or press against the walls of an artery and/or valve. In some embodiments, the frame 321 may comprise one or more arms, barbs, sutures, suture-engagement features, corkscrew-type or other tissue-engagement features, or the like.

In some embodiments, the cardiac implant structure 320 is physically integrated with and/or connected to a sensor device 310. The sensor device 310 may be, for example, a pressure sensor, or other type of sensor. In some embodiments, the sensor 310 comprises one or more transducers 312, such as one or more pressure transducers, as well as certain control circuitry 314, which may be embodied in, for example, an application-specific integrated circuit (ASIC). The sensor device 310 can have a generally soft structure and/or may be moldable to fit into various-sized openings of the cardiac implant structure 420. For example, the sensor device 310 may be at least partially composed of a polymer and/or thin metals. The sensor device 310 can be secured to the implant structure 320 by certain sensor-retention structure 325 (e.g., posts), examples of which are disclosed in detail herein. The sensor device 310 and/or sensor-retention structure 325 can be secured/stabilized using a stabilizer, which may be integrated or associated with the sensor-retention structure 325 or other component of the sensor implant device 300.

The control circuitry 314 may be configured to process transmission signals received from the transducer 312 and/or communicate signals wirelessly through biological tissue using the antenna 318. The antenna 318 may comprise one or more coils (e.g., electrically conductive coils) or loops of conductive material, such as copper wire or the like. In some embodiments, at least a portion of the transducer 312, control circuitry 314, and/or the antenna 318 are at least partially disposed or contained within a sensor housing 316, which may comprise any type of material, and may advantageously be at least partially hermetically sealed. For example, a sheath may be used to at least partially cover the antenna 318 (e.g., may cover one or more coils of wire), transducer 312, and/or control circuitry 314. In some embodiments, the housing 316 may be at least partially flexible. For example, the housing may comprise polymer or other flexible structure/material, which may advantageously allow for folding, bending, or collapsing of the sensor 310 to allow for transportation thereof through a catheter or other introducing means. In some embodiments, the sensor housing 316 (e.g., the frame 321) is at least partially cylindrical in shape.

The transducer 312 may comprise any type of sensor means or mechanism. For example, the transducer 312 may be a force-collector-type pressure sensor. In some embodiments, the transducer 312 comprises a diaphragm, piston, bourdon tube, bellows, or other strain- or deflection-measuring component(s) to measure strain or deflection applied over an area/surface thereof. The transducer 312 may be associated with the housing 316, such that at least a portion thereof is contained within or attached to the housing 316. The term “associated with” is used herein according to its broad and ordinary meaning. With respect to sensor devices/components being “associated with” a shunt or other implant structure, such terminology may refer to a sensor device or component being physically coupled, attached, or connected to, or integrated with, the implant structure. That is, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

In some embodiments, the transducer 312 comprises or is a component of a piezoresistive strain gauge, which may be configured to use a bonded or formed strain gauge to detect strain due to applied pressure, wherein resistance increases as pressure deforms the component/material. The transducer 312 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 312 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon, and the like. In some embodiments, the transducer 312 comprises or is a component of an electromagnetic pressure sensor, which may be configured to measure the displacement of a diaphragm by means of changes in inductance, linear variable displacement transducer (LVDT) functionality, Hall Effect, or eddy current sensing. In some embodiments, the transducer 312 comprises or is a component of a piezoelectric strain sensor. For example, such a sensor may determine strain (e.g., pressure) on a sensing mechanism based on the piezoelectric effect in certain materials, such as quartz.

In some embodiments, the transducer 312 comprises or is a component of a strain gauge. For example, a strain gauge embodiment may comprise a pressure sensitive element on or associated with an exposed surface of the transducer 312. In some embodiments, a metal strain gauge is adhered to a surface of the sensor, or a thin-film gauge may be applied on the sensor by sputtering or other technique. The measuring element or mechanism may comprise a diaphragm or metal foil. The transducer 312 may comprise any other type of sensor or pressure sensor, such as optical, potentiometric, resonant, thermal, ionization, or other types of strain or pressure sensors.

The efficacy of an implanted prosthetic heart valve may be measured based on the measurements of pressure, fluid flow through the valve, and/or other mechanisms that may provide indications of cardio output and/or heart function in general. Acute monitoring of heart/valve performance may be performed in a variety of ways, such as through the use of echo-based technologies (e.g., ultrasound, etc.) to measure the speed of fluid flow through the valve, which may be used to derive other calculations, such as pressure gradient, and the like. Imaging technologies (e.g., CT scan or X-ray) may provide information related to the opening/closing of heart valves, which may be used to determine blood volumes, etc.

When an individual has experienced compromised heart function over a period of time, transition to a new prosthetic heart valve may be somewhat prolonged. Therefore, although acute heart/valve monitoring may be performed during and immediately after surgery, continued monitoring of heart/valve function over a prolonged period of time post-surgery may be necessary or desirable. In addition, implant patients are often prescribed various medication dosages to assist in the recovery process. However, improper dosages may manifest in heart/valve complications that should be resolved as soon as possible.

Therefore, for at least these reasons, post-operative monitoring (e.g., continuous monitoring) over a period of time, such as for 15 days, 30 days, 45 days, 60 days, 90 days, or some other period post operation, may be desirable. For example, continued monitoring may provide the opportunity to intervene in the patient's recovery, such as by changing medication/dosage, before symptoms of malfunction manifest, and therefore earlier detection and response may be possible. Possible complications from heart valve implant surgery may include decreased ejection fraction, undesirable changes in pressure or pressure regulation malfunction, irregular heart rhythm (e.g., caused by surgical incisions), as well as other conditions. Certain embodiments provide a heart valve configured with one or more sensors for monitoring parameters related to such conditions, as well as a mechanism for communicating such information to one or more external systems and/or subsystems.

Embodiments of the present disclosure provide systems, devices, and methods for determining and/or monitoring fluid pressure and/or other physiological parameters or conditions in the left atrium using one or more implantable sensor devices, such as permanently implanted sensor devices. By placing a permanent sensor monitor device directly in the left atrium, embodiments of the present disclosure can advantageously allow physicians and/or technicians to gather real-time cardiac information, including left atrial pressure values and/or other valuable cardiac parameters.

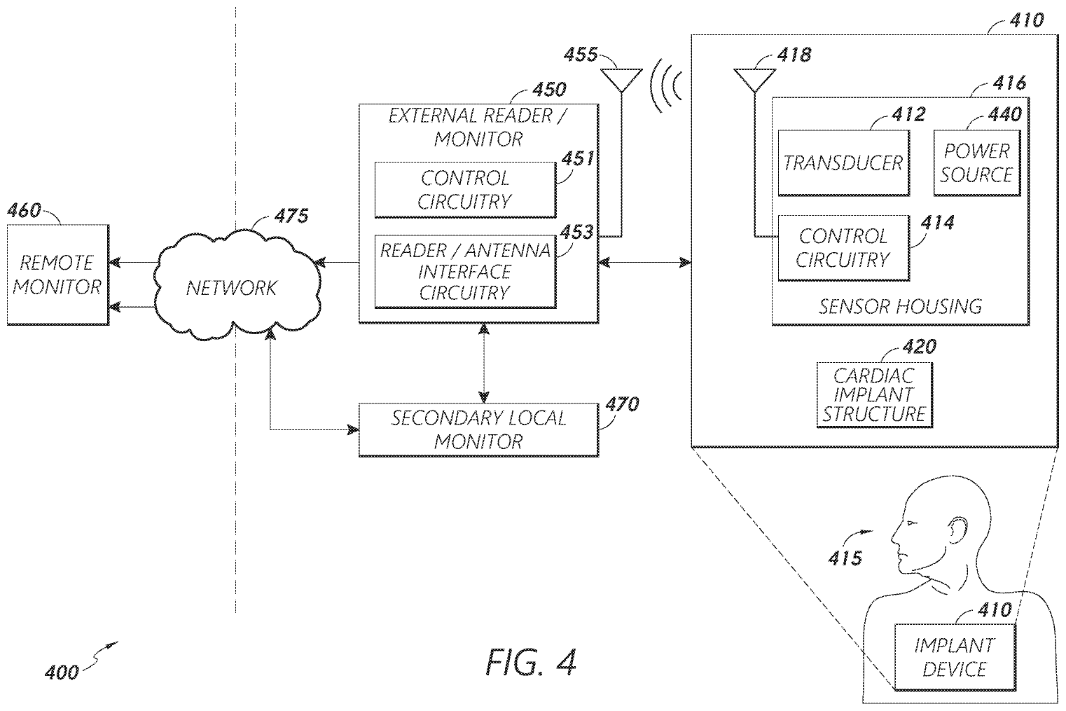

Disclosed solutions for implanting and maintaining sensor implant devices including certain stabilizer features may be implemented in connection with a pressure-monitoring system. FIG. 4 illustrates a system 400 for monitoring pressure and/or other parameter(s) associated with a patient 415 in accordance with embodiments of the present disclosure. Although the description of FIG. 4 and other embodiments herein is generally presented in the context of pressure monitoring, it should be understood that description of pressure sensing and pressure sensor stabilizing herein is applicable to sensing/stabilization of other types of sensors and sensing of other types of physiological parameters, wherein sensor devices used for such purposes are stabilized using certain stabilizer features.

The patient 415 can have a sensor implant device 410 (e.g., a pressure sensor implant device) implanted in, for example, the heart (not shown), or associated physiology, of the patient. For example, the sensor implant device 410 can be implanted at least partially within the left atrium of the patient's heart. The sensor implant device 410 can include one or more sensor transducers 412, such as one or more microelectromechanical system (MEMS) devices, such as MEMS pressure sensors, or the like.

In certain embodiments, the monitoring system 400 can comprise at least two subsystems, including an implantable internal subsystem or sensor implant device 410 that includes the sensor transducer(s) 412 (e.g., MEMS pressure sensor(s)), as well as control circuitry 414 comprising one or more microcontroller(s), discrete electronic component(s), and one or more power and/or data transmitter(s) 418 (e.g., antennae coil). The monitoring system 400 can further include an external (e.g., non-implantable) subsystem that includes an external reader 450 (e.g., coil), which may include a wireless transceiver that is electrically and/or communicatively coupled to certain control circuitry. In certain embodiments, both the internal and external subsystems include a corresponding antenna for wireless communication and/or power delivery through patient tissue disposed therebetween. The sensor implant device 410 can be any type of implant device.

The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coating of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

Certain details of the sensor implant device 410 are illustrated in the enlarged block shown. The sensor implant device 410 can comprise implant structure 420 (e.g., an anchor structure) as described herein. For example, the implant structure 420 can include one or more shunt-type implants/anchors for anchoring in a cardiac tissue wall, as described in greater detail below. The implant structure 420 can further comprise one or more arm structures that physically hold/secure the implant structure 420 to a tissue wall, for example. Although certain components are illustrated in FIG. 4 as part of the sensor implant device 410, it should be understood that the sensor implant device 410 may only comprise a subset of the illustrated components/modules and can comprise additional components/modules not illustrated. The sensor implant device 410 includes one or more sensor transducers 412, which can be configured to provide a response indicative of one or more physiological parameters of the patient 415, such as atrial pressure and/or volume. Although pressure transducers are described, the sensor transducer(s) 412 can comprise any suitable or desirable types of sensor transducer(s) for providing signals relating to physiological parameters or conditions associated with the sensor implant device 410.

The sensor transducer(s) 412 can comprise one or more MEMS sensors, optical sensors, piezoelectric sensors, electromagnetic sensors, strain sensors/gauges, accelerometers, gyroscopes, and/or other types of sensors, which can be positioned in the patient 415 to sense one or more parameters relevant to the health of the patient. The transducer 412 may be a force-collector-type pressure sensor. In some embodiments, the transducer 412 comprises a diaphragm, membrane, piston, bourdon tube, bellows, or other strain- or deflection-measuring component(s) to measure strain or deflection applied over an area/surface thereof. The transducer 412 may be associated with a sensor housing 416, such that at least a portion thereof is contained within, or attached to, the housing 416.

In some embodiments, the transducer 412 comprises or is a component of a piezoresistive strain gauge, which may be configured to use a bonded or formed strain gauge to detect strain due to applied pressure, wherein resistance increases as pressure deforms the component/material. The transducer 412 may incorporate any type of material, including but not limited to silicon (e.g., monocrystalline), polysilicon thin film, bonded metal foil, thick film, silicon-on-sapphire, sputtered thin film, and/or the like.

In some embodiments, the transducer 412 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicon or other semiconductor, and the like. In some embodiments, the transducer 412 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some embodiments, the transducer 412 comprises or is a component of a piezoelectric strain sensor. For example, such a sensor may determine strain (e.g., pressure) on a sensing mechanism based on the piezoelectric effect in certain materials, such as quartz.

In some embodiments, the transducer 412 comprises or is a component of a strain gauge. For example, a strain gauge embodiment may comprise a pressure sensitive element on or associated with an exposed surface of the transducer 412. In some embodiments, a metal strain gauge is adhered to the sensor surface, or a thin-film gauge may be applied on the sensor by sputtering or other technique. The measuring element or mechanism may comprise a diaphragm or metal foil. The transducer 412 may comprise any other type of sensor or pressure sensor, such as optical, potentiometric, resonant, thermal, ionization, or other types of strain or pressure sensors.

In some embodiments, the transducer(s) 412 is/are electrically and/or communicatively coupled to the control circuitry 414, which may comprise one or more application-specific integrated circuit (ASIC) microcontrollers or chips. The control circuitry 414 can further include one or more discrete electronic components, such as tuning capacitors or the like.

In certain embodiments, the sensor transducer(s) 412 can be configured to generate electrical signals that can be wirelessly transmitted to a device outside the patient's body 415, such as the illustrated external reader 450 (e.g., a local monitor system). In order to perform such wireless data transmission, the sensor implant device 410 can include radio frequency (RF) transmission circuitry, such as a signal processing circuitry and an antenna/data transmitter 418. The antenna 418 can comprise an internal antenna coil or other structure implanted within the patient. The control circuitry 414 may comprise any type of transducer circuitry configured to transmit an electromagnetic signal, wherein the signal can be radiated by the antenna 418, which may comprise one or more conductive wires, coils, plates, or the like. The control circuitry 414 of the sensor implant device 410 can comprise, for example, one or more chips or dies configured to perform some amount of processing on signals generated and/or transmitted using the sensor implant device 410. However, due to size, cost, and/or other constraints, the sensor implant device 410 may not include independent processing capability in some embodiments.

The wireless signals generated by the sensor implant device 410 can be received by the external reader 450, which can include a transceiver module 453 configured to receive the wireless signal transmissions from the sensor implant device 410, which is disposed at least partially within the patient 415. The external reader 450 can receive the wireless signal transmissions and/or provide wireless power using an external antenna 455, such as a wand device. The transceiver 453 can include radio-frequency (RF) front-end circuitry configured to receive and amplify the signals from the sensor implant device 410, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The transceiver 453 can further be configured to transmit signals over a network 475 to a remote monitor 460 (e.g., a remote subsystem or device). The RF circuitry of the transceiver 453 can further include one or more of digital-to-analog converter (DAC) circuitry, power amplifiers, low-pass filters, antenna switch modules, antennas or the like for treatment/processing of transmitted signals over the network 475 and/or for receiving signals from the sensor implant device 410. In certain embodiments, the external reader 450 includes control circuitry 451 for performing processing of the signals received from the sensor implant device 410. The external reader 450 can be configured to communicate with the network 475 according to a known network protocol, such as Ethernet, Wi-Fi, or the like. In certain embodiments, the external reader 450 is a smartphone, laptop computer, or other mobile computing device, or any other type of computing device.

In certain embodiments, the sensor implant device 410 includes some amount of volatile and/or non-volatile data storage. For example, such data storage can comprise solid-state memory utilizing an array of floating-gate transistors, or the like. The control circuitry 414 may utilize data storage for storing sensed data collected over a period of time, wherein the stored data can be transmitted periodically to the external reader 450 or other external subsystem. In certain embodiments, the sensor implant device 410 does not include any data storage. The control circuitry 414 is configured to facilitate wireless transmission of data generated by the sensor transducer(s) 412, or other data associated therewith. The control circuitry 414 may further be configured to receive input from one or more external subsystems, such as from the external reader 450, or from a remote monitor 460 over, for example, the network 475. For example, the sensor implant device 410 may be configured to receive signals that at least partially control the operation of the sensor implant device 410, such as by activating/deactivating one or more components or sensors, or otherwise affecting operation or performance of the sensor implant device 410.

The one or more components of the sensor implant device 410 can be powered by one or more power sources 440. Due to size, cost and/or electrical complexity concerns, it may be desirable for the power source 440 to be relatively minimalistic in nature. For example, high-power driving voltages and/or currents in the sensor implant device 410 may adversely affect or interfere with operation of the heart or other anatomy associated with the implant device. In certain embodiments, the power source 440 is at least partially passive in nature, such that power can be received from an external source wirelessly by passive circuitry of the sensor implant device 410. Examples of wireless power transmission technologies that may be implemented include but are not limited to short-range or near-field wireless power transmission, or other electromagnetic coupling mechanism(s). For example, the external reader 450 may serve as an initiator that actively generates an RF field that can provide power to the sensor implant device 410, thereby allowing the power circuitry of the implant device to take a relatively simple form factor. In certain embodiments, the power source 440 can be configured to harvest energy from environmental sources, such as fluid flow, motion, pressure, or the like. Additionally or alternatively, the power source 440 can comprise a battery, which can advantageously be configured to provide enough power as needed over the relevant monitoring period.

In some embodiments, the external reader 450 can serve as an intermediate communication device between the sensor implant device 410 and the remote monitor 460. The external reader 450 can be a dedicated external unit designed to communicate with the sensor implant device 410. For example, the external reader 450 can be a wearable communication device, or other device that can be readily disposed in proximity to the patient 415 and/or sensor implant device 410. The external reader 450 can be configured to continuously, periodically, or sporadically interrogate the sensor implant device 410 in order to extract or request sensor-based information therefrom. In certain embodiments, the external reader 450 comprises a user interface, wherein a user can utilize the interface to view sensor data, request sensor data, or otherwise interact with the external reader 450 and/or sensor implant device 410.

The system 400 can include a secondary local monitor 470, which can be, for example, a desktop computer or other computing device configured to provide a monitoring station or interface for viewing and/or interacting with the monitored cardiac data. In an embodiment, the external reader 450 can be a wearable device or other device or system configured to be disposed in close physical proximity to the patient and/or sensor implant device 410, wherein the external reader 450 is primarily designed to receive/transmit signals to and/or from the sensor implant device 410 and provide such signals to the secondary local monitor 470 for viewing, processing, and/or manipulation thereof. The external reader 450 can be configured to receive and/or process certain metadata from or associated with the sensor implant device 410, such as device ID or the like, which can also be provided over the data coupling from the sensor implant device 410.

The remote monitor 460 can be any type of computing device or collection of computing devices configured to receive, process and/or present monitor data received over the network 475 from the external reader 450, secondary local monitor 470, and/or sensor implant device 410. For example, the remote monitor 460 can advantageously be operated and/or controlled by a healthcare entity, such as a hospital, doctor, or other care entity associated with the patient 415.

In certain embodiments, the antenna 455 of the external reader 450 comprises an external coil antenna that is matched and/or tuned to be inductively paired with the antenna 418 of the sensor implant device 410. In some embodiments, the sensor implant device 410 is configured to receive wireless ultrasound power charging and/or data communication between from the external reader 450. As referenced above, the external reader 450 can comprise a wand or other hand-held reader.

In some embodiments, at least a portion of the transducer 412, control circuitry 414, power source 440 and/or the antenna 418 is at least partially disposed or contained within the sensor housing 416, which may comprise any type of material, and may advantageously be at least partially hermetically sealed. For example, the housing 416 may comprise glass or other rigid material in some embodiments, which may provide mechanical stability and/or protection for the components housed therein. In some embodiments, the housing 416 is at least partially flexible. For example, the housing may comprise polymer or other flexible structure/material, which may advantageously allow for folding, bending, or collapsing of the sensor implant device 410 to allow for transportation thereof through a catheter or other percutaneous introducing means.

The sensor housing 416 can be secured to certain sensor-retention structure (e.g., posts), which may be physically coupled to and/or integrated with the cardiac implant structure 420 (e.g., a valve frame). For example, in some embodiments, the sensor-retention structure is integrated with a post extending from the implant structure 420. Such posts may in some cases be secondary elements which may be added to an existing implant structure 420. For example, a post may be added to extend from one or more struts of a valve frame.

The sensor implant device 410 may be implanted in any location in the body the patient 415. In some embodiments of the present disclosure, the sensor implant device 410 is advantageously implanted in the heart of the patient 415, such as in or near the aortic valve of the heart, as described in detail herein. Sensor implant devices in accordance with one or more embodiments of the present disclosure may be implanted using transcatheter procedures, or any other percutaneous procedures. Alternatively, sensor implant devices in accordance with aspects of the present disclosure may be placed during open-heart surgery (e.g., sternotomy), mini-sternotomy, and/or other surgical operation.

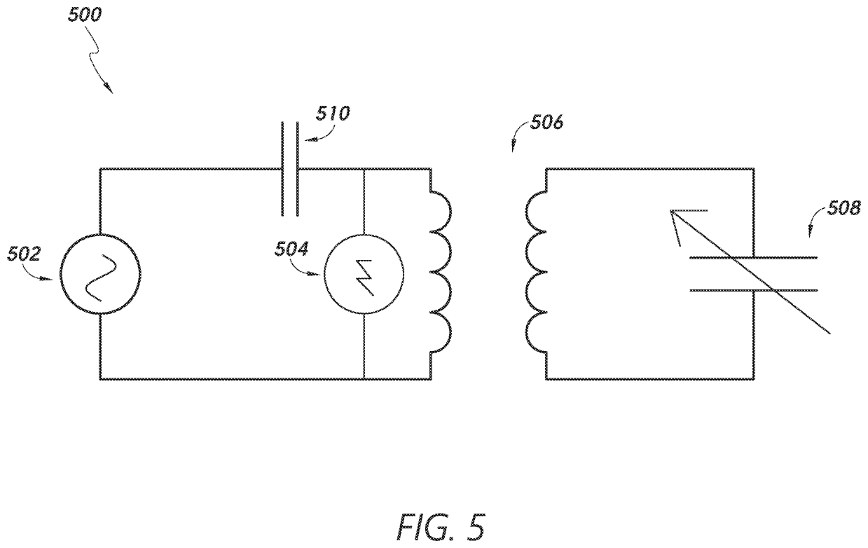

FIG. 5 provides a schematic drawing of an example circuit 500 for one or more sensors as described herein which may be attached to an artificial valve for gathering and/or wirelessly transmitting data to an external receiver (e.g., outside the body). In some embodiments, a circuit may comprise a voltage source (e.g., an AC voltage source) 502, an oscilloscope 504, a transformer 506 (e.g., comprising two inductor coils), a variable capacitor 508, and/or another capacitor 510. The oscilloscope 504 may be used to display the shape of electrical signals transmitted from the circuit 500.

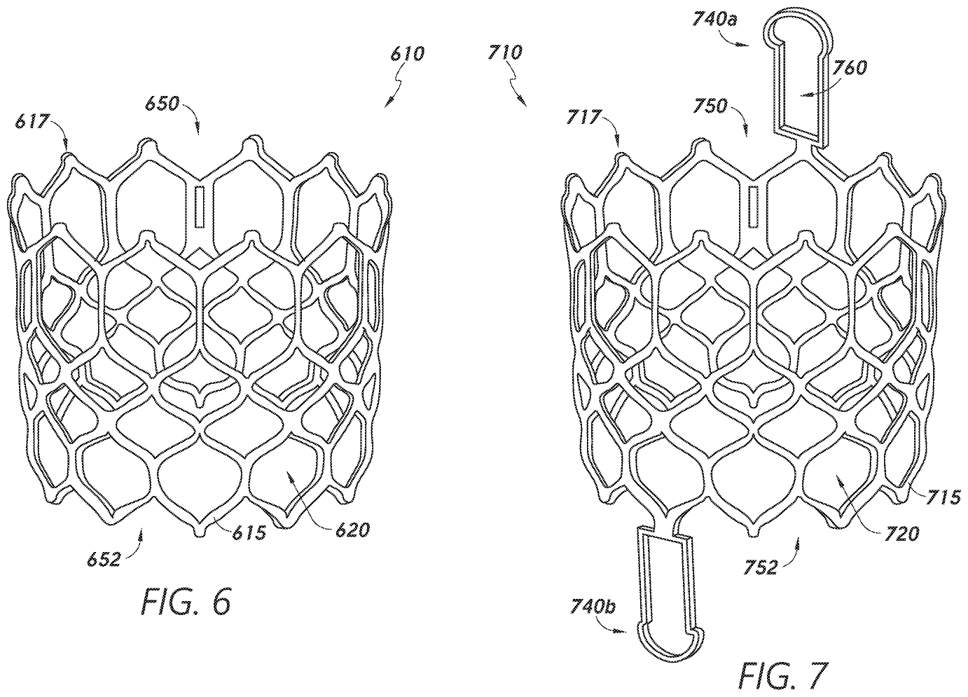

FIG. 6 depicts an example frame 610 comprising a network of struts 615 forming one or more cells 620. The frame 610 may form a lumen through a middle portion of the frame 610 and/or passing from a first end portion 650 to a second end portion 652, with a first opening at the first end portion 650 and/or a second opening at the second end portion 652. The dimensions and/or shape of the frame 610 may vary based on the particular application. In some cases, blood can freely pass through the cells 620 of the frame 610. In some embodiments, the frame 610 may comprise an inner and/or outer lining and/or other layers which can prevent the flow of blood through the cells 620.

The network of struts 615 forming the frame 610 may form one or more end points 617 at the first end portion 650 and/or second end portion 652 of the frame 610. The one or more end points 617 may be positioned at an inflow portion (e.g., the first portion 650) and/or at an outflow portion (e.g., the second portion 652) of the frame 610. The frame 610 may be configured to allow blood flow through the frame 610 and/or to otherwise operate as a prosthetic valve of a heart.

In some embodiments, the frame 610 may be configured to be crimped to facilitate introduction of the frame 610 into a patient's body and/or to a target location within the body. Crimping may involve reducing a diameter of the frame and/or increasing a length of the frame (e.g., increasing a distance between the first portion 650 and the second portion 652).

FIG. 7 illustrates a prosthetic valve including a frame 710 and one or more posts 740 extending from the frame 710. While FIG. 7 shows two posts 740 extending from the frame 710, prosthetic valves may include any number of posts 740 extending from a frame 710. Moreover, while a first post 740a is shown at a top portion 750 of the frame 710 and a second post 740b is shown at a bottom portion 752 of the frame 710, the frame 710 may include any number of posts 740 extending from the top portion 750 of the frame 710 and/or any number of posts 740 extending from the bottom portion 752 of the frame 710. For example, the frame 710 may include four posts 740 extending from the top portion 750 and four posts 740 extending from the bottom portion 752.

In some embodiments, a post 740 may comprise a wire form forming an islet 760 (i.e., sensor receptor) having any suitable shape and/or size. The islet 760 may be configured to receive one or more sensors and/or associated devices. The posts 740 may be configured to position one or more sensors at or near the top portion 750 of the frame 710 and/or at or near the bottom portion 752 of the frame 710. In this way, the one or more sensors may be configured to determine a pressure differential between the top portion 750 of the frame 710 and the bottom portion 752 of the frame 710.

The one or more posts 740 may be configured to position one or more sensors in direct contact with blood flow around and/or through the frame 710. Moreover, the one or more sensors may be configured to extend from one or more end portions of the frame 710 such that the one or more posts 740 do not interfere with the functionality of the frame 710. For example, the frame 710 may be configured to be crimped and/or otherwise compacted to be fit through a catheter and or other delivery device. The one or more posts 740 may be configured to facilitate and/or allow for crimping of the frame 710.

In some embodiments, one or more posts 740 may be added to an existing frame 710. For example, one or more posts 740 may function as a secondary element and/or “backpack” feature which may be configured to be attached to and/or woven throughout the frame 710. In this way, the one or more posts 740 may advantageously be configured for use with various types of frames 710. Moreover, the positioning of the posts 740 at one or more end portions of the frame 710 without extending within a central lumen of the frame may allow the posts to function without altering the functionality of the frame 710.

As shown in FIG. 7, one or more posts 740 may be configured to extend from end portions 717 of one or more struts 715 of a frame. For example, the frame 710 may comprise one or more cells 720 forming empty space between struts 715 of the frame 710. The cells 720 may have any shape including the generally hexagonal shape shown in FIG. 7. The struts 715 surrounding the cells 720 may form various end portions 717 from which one or more posts 740 may be configured to extend.

The frame 710 and/or one or more posts 740 may be formed through use of a laser cut process. For example, the one or more posts 740 may be added to a flat-pattern frame 710 before the frame 710 is cut into a tubing form (e.g., a 23 mm tubing) shown in FIGS. 6 and 7.

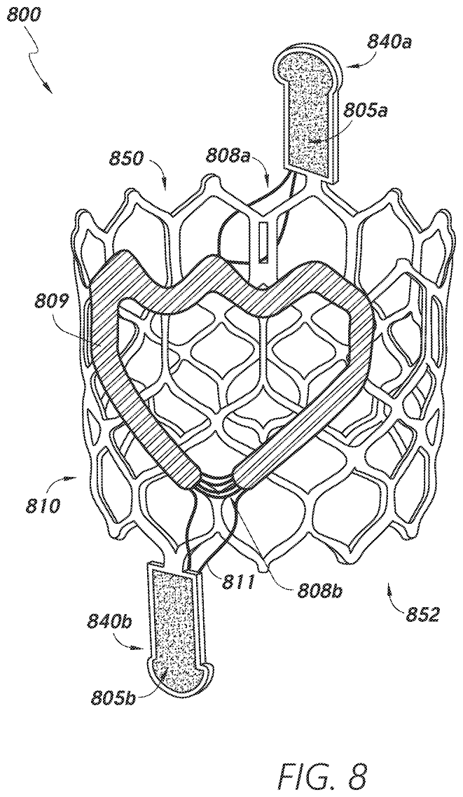

FIG. 8 illustrates a prosthetic valve 800 comprising a frame 810 and one or more sensors 805 at one or more posts 840 extending from the frame 810. In some embodiments, one or more sensors 805 may have a generally soft structure. For example, a sensor 805 may be composed of a polymer which may have a reduced risk of damaging the surrounding tissue relative to metallic devices. In some embodiments, one or more sensors 805 may be composed at least partially of very thin metal such that the structure of the one or more sensors 805 is relatively soft.

One or more electrically conductive coils 808 may be attached to each of the one or more sensors 805. The one or more coils 808 may be configured to pass along an inner and/or outer surface of the frame 810 and/or may otherwise be configured to attach to the frame 810. One or more coils 808 may be configured to form an internal wrapping at the inner and/or outer surface of the frame 810 and/or connect to one or more sensors at a first portion 850 (e.g., an inflow portion) and/or a second portion 852 (e.g., an outflow portion) of the frame 810. In some embodiments, one or more coils 808 may be configured to at least partially cover a circumference of the frame 810 structure. For example, a first coil 808 may cover a circumference at or near the first portion 850 of the frame 810. In some embodiments, separate coils 808 may be used and/or may be connected to separate sensors 805. For example, a first coil 808a may be configured to connect to a first sensor 805a at the first portion 850 of the frame 810 while a second coil 808b may connect to a second sensor 805b at a second portion 852 of the frame 810. One or more sensors 805 at the first portion 850 of the frame 810 may be configured to work in parallel with one or more sensors 805 at the second portion 852 of the frame 810.

Sensor data collected by one or more sensor(s) 805 may be transmitted to an external receiver (not shown) using a transmitter assembly. The transmitter assembly may include the one or more electrically conductive coils 808 electrically coupled to the one or more electronic sensors 805 and/or circuits. The one or more coil 808 may be configured to provide power to the sensors/circuits 805, transmit electromagnetic signals to an external receiver, and/or receive power/data therefrom. For example, a coil 808 may operate as an antenna for receiving wireless power and/or for transmitting electromagnetic signals. In certain embodiments, the transmitter assembly may be embedded in, or integrated with, the frame 810. For example, the transmitter assembly may be at least partially nested within a recess, channel, or cavity of the frame 810. By embedding the transmitter assembly in an outer portion of the frame 810, the sensor 805 may be configured to effectively transfer electromagnetic signals to a remote receiver.

In certain embodiments, the transmission assembly, including the one or more sensors 805, may be configured to communicate power and/or data according to inductive coupling, resonant inductive coupling (e.g., RFID), capacitive coupling, or the like. For example, the transmission assembly may be configured to transmit information relating to sensed biological or device parameter(s), as well as data identifying one or more of the valves (e.g., make, model, identification number, serial number) and/or the patient (e.g., name, identification number, patient identifier).

The transmitter assembly may have a shape that generally conforms to the shape of a portion of the frame 810 assembly. The one or more coils 808 may comprise one or more conductive wires wrapped around a circumferential path of the assembly. In certain embodiments, the one or more coils 808 may be at least partially covered with a sheath or covering 809, which may provide electrical, thermal, and/or physical isolation between the coils 808 and external components or structures of the frame 810 with which the assembly is associated.

The one or more coils 808 may be electrically coupled via one or more leads 811 to the one or more sensors 805. The coils 808 may be coupled to any number of sensors 805 attached to and/or extending from the frame 810. The one or more sensors 805 may be assembled to receive power wirelessly and/or transmit sensor and/or other data wirelessly using the one or more coils 808 as an antenna.

Each of a first sensor 805a and a second sensor 805b may be coupled to separate coils 808 (e.g., a first coil 808a and a second coil 808b, respectively). The first coil 808a and the second coil 808b may not be attached to each other. Moreover, while a second sheath 809 is not shown in FIG. 8, the first coil 808a may be at least partially covered by a sheath 809 to at least partially cover and/or provide isolation to the first coil 808a.

The first sensor 805a and the second sensor 805b may be configured to measure differential pressure using pressure measurements on either side of the valve 800. Through use of sensors at the first portion 850 of the frame 810 and at the second portion 852 of the frame 810, the valve 800 may be configured to provide a calibration-free reading of the pressure gradient across the valve 800. In some embodiments, the first sensor 805a and the second sensor 805b may be connected through use of a fluidic and/or electrical connection. For example, a substrate and/or one or more coils may be configured to connect the first sensor 805a to the second sensor 805b. However, the first sensor 805a may not necessarily be physically connected to the second sensor 805b and/or the first sensor 805a and/or second sensor 805b may be configured to wirelessly communicate measurements to an external receiver.

In some embodiments, the one or more sensors 805 and/or other components may be configured to perform some amount of signal processing for signal transmission, such as signal filtering, amplification, mixing, and/or the like. For example, the one or more sensors 805 may include one or more processors, data storage devices, data communication busses, and/or the like.

The devices, systems and methods disclosed herein may be used for identifying symptoms or conditions indicating potential heart or implant failure issues in patients that have received a prosthetic heart valve implant, or other implant device. Some implementations provide for the use of one or more sensors 805 to sense and/or transmit various measurements (e.g., blood pressure) and valve function in a heart valve device.

One or more sensors 805 may be applied to a wireform or stent component of the prosthetic valve 800. Although sensors 805 for measuring blood pressure are discussed in detail herein, other sensors may be used, such as strain gauges, accelerometers, gyroscopes, optical sensors, or the like. The data provided by, or derived from, one or more sensor(s) 805 in an implanted heart valve may be used to alert a patient or health care provider of a change in the patient's heart rate or blood pressure and may provide an early indication of a change in heart function. As described above, patients who undergo a prosthetic heart valve implant operation can sometimes have post-implant heart failure related morbidity/mortality. Heart valve sensor devices and wireless data transmission functionality as disclose herein may be able to provide early information regarding heart function and thus allow for earlier intervention for patients.

In some embodiments, the valve 800 may include a first post 840a configured to receive a first sensor 805a and/or may include a second post 840b configured to receive a second sensor 805b. The first post 840a may be configured to extend from an end point of the frame 810 at an inflow portion of the frame 810 and/or the second post 840b may be configured to extend from an end point of the frame 810 at an outflow portion of the frame 810. In response to crimping of the valve 800, a distance between the first post 840a and the second post 840b may increase. In this way, the first sensor 805a and the second sensor 805b may advantageously be configured to expand with the valve 800 and may not restrict the movement of the valve 800 (e.g., during a delivery process involving crimping of the valve 800).

Some amount of power may be necessary for powering the one or more sensors 805. For example, an excitation voltage applied to input leads of the one or more sensors 805 may be provided from wireless power transfer, local power harvesting, local power storage, or other power generation and/or supply system. In some embodiments, one or more piezoelectric crystals may be used to generate power, which may be stored in a power storage device, such as a capacitor or the like. The voltage reading of the one or more sensors 805 may be taken from one or more of the output leads 811. The frame 810 may comprise signal processing circuitry (not shown) for performing preprocessing on the sensor signal, such as filtering, signal amplification, or the like.

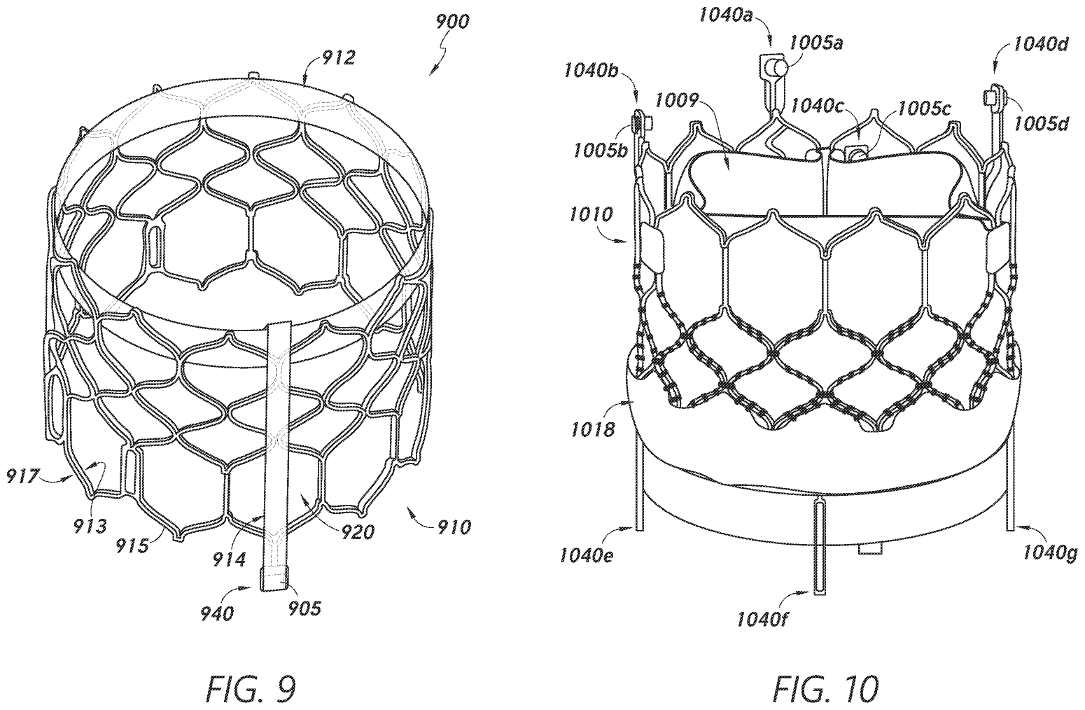

FIG. 9 illustrates another valve comprising a frame 910 and a substrate band 912 wrapped at least partially about a circumference at or near a first portion of the frame 910. In some embodiments, the substrate band 912 may be situated at least partially along an inner surface 913 of the frame as shown in FIG. 9. However, the substrate band 912 may additionally or alternatively be situated at an outer surface 917 of the frame 910. In some embodiments, the substrate band 912 may comprise a partial circular form (e.g., a semi-circle). The substrate band 912 may be sutured to the frame 910. In some embodiments, one or more coils may be sutured to the substrate 912.

One or more substrate extensions 914 may extend from the substrate band 912. For example, the substrate band 912 may be situated at or near a first portion (e.g., an inflow portion) of the frame 910 and a substrate extension 914 may be configured to extend axially from the substrate band 912 to a second portion (e.g., an outflow portion) of the frame. One or more sensors 905 may be configured to attach to an end portion of the substrate extension 914 such that the one or more sensors 905 may be configured to be situated at or near the second portion of the frame 910 while one or more sensors 905 attached to the substrate band 912 may be configured to be situated at the first portion of the frame 910. While only a single substrate extension 914 is shown in FIG. 9, any number of substrate extensions 914 may be included. For example, four substrate extensions 914 may extend from the substrate band 912, with each substrate extension 914 configured to deploy at least one sensor 905 at the second portion of the frame 910. The one or more substrate extensions 914 may be configured to pass along an inner surface 913 and/or outer surface 917 of the frame 910 and/or may be configured to attach to one or more struts 915 of the frame. In some embodiments, a substrate extension 914 may be configured to align with a post 940 of the frame 910 such that a sensor 905 attached to the substrate extension 914 may be configured to be situated within an islet of the post 940.

In some embodiments, a first sensor 905 situated at or near the first portion of the frame 910 may be powered by and/or may wirelessly transmit via a separate circuit (e.g., an LC resonant circuit) from a circuit used by a second sensor 905 at or near a second portion of the frame 910.

The substrate band 912 may comprise various contact lines. In some embodiments, the substrate band 912 may be configured to create a conduction path between multiple sensors, antennas, and/or other components. One or more substrate extensions 914 may be configured to further extend the conduction path from an inflow portion of the valve 900 to an outflow portion of the valve 900. In some embodiments, one or more substrate extensions 914 may be configured to pass at least partially over one or more cells 920 of the frame 910. For example, the one or more substrate extensions 914 may be configured to extend over empty space between struts 915 of the frame 910. At points where the one or more substrate extensions 914 pass over struts 915 of the frame 910, the one or more substrate extensions 914 may be configured to contact and/or attach to the frame 910.

FIG. 10 illustrates a valve comprising a frame 1010 and a skirt 1018 wrapped at least partially around an inner surface and/or outer surface of the frame 1010. In some embodiments, the skirt 1018 may be configured to prevent ingrowth of tissue through the cells of the frame 1010.

The valve may comprise any number of sensors 1005 and/or posts 1040. For example, the valve may comprise a first sensor 1005a, a second sensor 1005b, a third sensor 1005c, and a fourth sensor 1005d spaced circumferentially around a first portion of the valve. Each of the first sensor 1005a, second sensor 1005b, third sensor 1005c, and fourth sensor 1005d may be situated at and/or within a first post 1040a, second post 1040b, third post 1040c, and/or fourth post 1040d, respectively. The valve may further comprise a fifth post 1040e, sixth post 1040f, seventh post 1040g, and/or eighth post (not shown) at a second portion of the valve. Additional sensors 1005 may be situated at and/or within the posts 1040 at the second portion of the valve.

In some embodiments, the valve may comprise one or more prosthetic leaflets 1009 configured to replace missing and/or dysfunctional leaflets within the patient's body. The one or more prosthetic leaflets 1009 may be configured to cover at least a portion of an internal lumen of the valve.

The frame 1010 of the valve may be configured to support the one or more posts 1040 extending from the frame 1010. In some embodiments, the one or more posts 1040 may be configured to be attached to the frame 1010. When the valve is crimped, the one or more posts 1040 and/or one or more sensors 1005 may be configured to move with the frame.

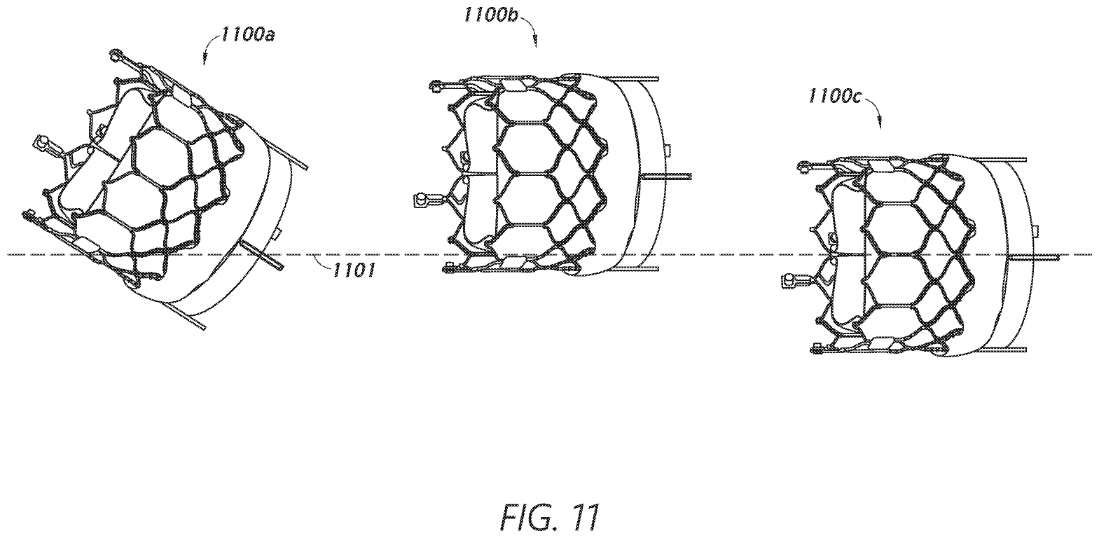

FIG. 11 illustrates how a valve alignment can be altered as a result of breathing or other chest movement of a patient. In some cases, successful data transfer between one or more sensors at a valve may require a parallel configuration with a remote antenna (e.g., positioning along and/or in parallel with a given line 1101 of communication). A first valve 1100a is shown not in parallel with the line 1101 of communication, a second valve 1100b is shown displaced from the line 1101 of communication, and a third valve 1100c is shown in parallel and in line with the line 1101 of communication.

Data transmissions can be unsuccessful when there is not a clear line of communication between a sensor antenna at a valve 1100 and a receiver. Accordingly, when a sensor is positioned within a frame of the valve 1100, communications may be unsuccessful at least in certain alignments due to portions of the valve blocking the wireless data path.

Some embodiments of the present disclosure advantageously provide sensor receptors which may be positioned to extend one or more sensors away from a frame of the valve 1100. For example, a valve 1100 may comprise one or more posts configured to position one or more sensors at or beyond an outflow portion and/or an inflow portion of the valve 1100. By positioning one or more sensors distally from the frame of the valve 1100, transmissions from the one or more sensors can be improved in various alignments of the valve 1100.

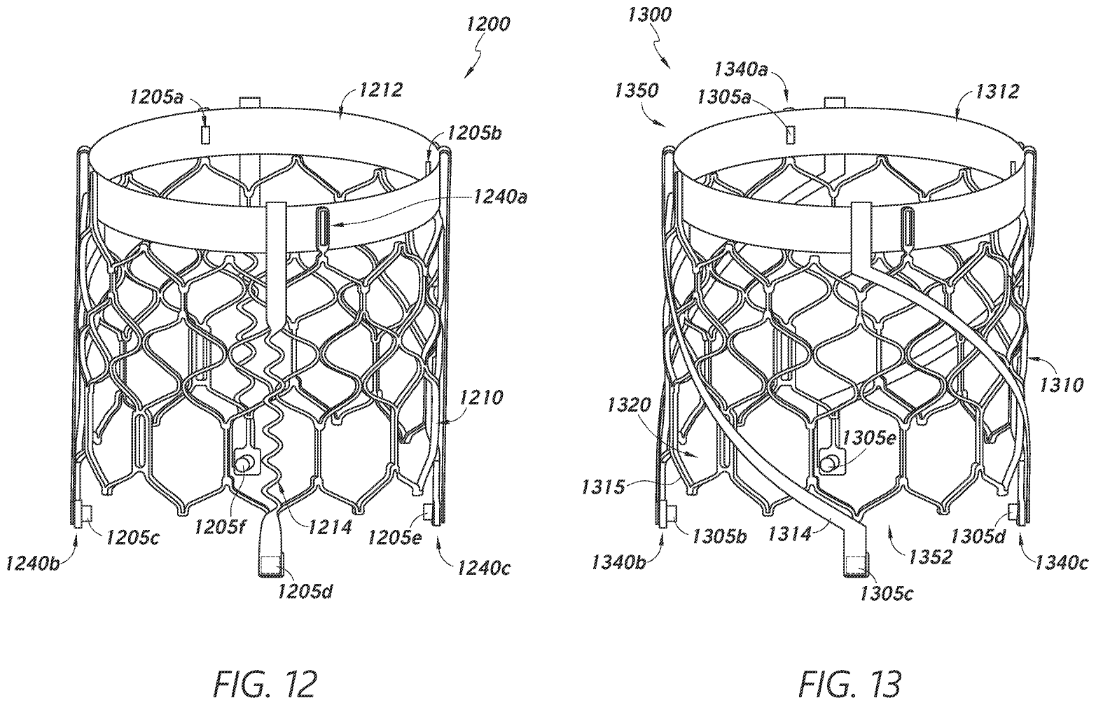

FIG. 12 illustrates a frame 1210 comprising a substrate band 1212 and one or more substrate extensions 1214 having an extendible structure. The one or more substrate extensions 1214 may be configured to extend generally axially from the substrate band 1212. However, the one or more substrate extensions 1214 may have a generally non-linear (e.g., serpentine and/or zigzag) structure in which the substrate extensions 1214 comprise one or more bends to allow the substrate extensions 1214 to be extended and/or compressed. The extendibility of the substrate extensions 1214 may advantageously allow the one or more sensors 1205 of the second portion of the frame 1210 to extend further from the substrate band 1212 as the frame 1210 is crimped and/or as the frame 1210 itself is extended in response to crimping.

As shown in FIG. 12, the valve 1200 may comprise multiple sensors 1205 at or near the first portion of the valve 1200 and/or at or near the second portion of the valve 1200. For example, the valve 1200 may comprise at least a first sensor 1205a at the first portion. The valve 1200 may additionally comprise a second sensor 1205b at the first portion and/or a third sensor 1205c, fourth sensor 1205d, fifth sensor 1205e, and/or sixth sensor 1205f at the second portion. In some embodiments, the valve 1200 may comprise four sensors 1205 at or near the first portion. The use of multiple sensors at the first portion and/or second portion may allow for improved detection of variations in pressure and/or flow around the periphery of the valve 1200. In some embodiments, multiple sensors 1205 may be connected to a common coil and/or to separate coils. For example, the second sensor 1205b, third sensor 1205c, fourth sensor 1205d, and/or fifth sensor 1205e may be connected to the same coil to determine an average measurement reading and/or may each be coupled to different coils to determine distributed measurements around the periphery of the valve 1200.

The valve 1200 may include any number of posts 1240, including a first post 1240a, at a first end portion of the valve 1200 and/or at or near the substrate band. As shown in FIG. 12, the first post 1240a may be configured to extend at least partially along the substrate band 1212. A sensor 1205 may be configured to be situated within the first post 1240a while simultaneously attached and/or coupled to the substrate band 1212. The valve 1200 may further include a second post 1240b and/or a third post 1240c extending from end points of a second end portion of the valve 1200. The third sensor 1205c, fourth sensor 1205d, fifth sensor 1205e, and/or sixth sensor 1205f may be configured to be situated within a corresponding post and/or coupled to a substrate extension 1214 extending form the substrate band 1212.

In response to crimping of the valve 1200 (e.g., the valve 1200 increasing in length to increase a distance between the substrate band 1212 at a first end portion of the valve 1200 and the second post 1240b at a second end portion of the valve 1200), a substrate extension 1214 may be configured to become more linear. For example, a curvature of the substrate extension 1214 may be reduced. In some embodiments, the substrate extension 1214 may be at least partially composed of a flexible and/or elastic material such that the substrate extension 1214 can mold (e.g., extend) in response to crimping and/or lengthening of the valve 1200 and/or to return to the original form shown in FIG. 12 following the crimping process (e.g., in response to expansion of the valve 1200).

FIG. 13 illustrates another example valve 1300 including a frame 1310, a substrate band 1312, and one or more substrate extensions 1314 configured to extend generally diagonally and/or at an approximately 45-degree angle from the substrate band 1312 at a first portion 1350 of the frame 1310 to a second portion 1352 of the frame 1310. The one or more substrate extensions 1314 may be configured to pass at least partially circumferentially about an outer surface and/or inner surface of the frame 1310 such that an end portion of a substrate extension 1314 may not be situated directly below the point at which the substrate extension 1314 attaches to substrate band 1312. The one or more substrate extensions 1314 may be at least partially flexible and/or may be curved at least partially to allow the substrate extensions 1314 to be extended and/or contracted in response to crimping and/or expansion of the frame 1310.

In some embodiments, one or more substrate extensions 1314 may be configured to have a generally “serpentine” structure and/or may form generally thin elongate structures extending from the substrate band 1312. One or more substrate extensions 1314 may be configured to at least partially wrap around an outer surface and/or an inner surface in a generally diagonal direction that is between an axial direction (e.g., a direct line from the first portion 1350 to the second portion 1352) and a circumference direction (e.g., in line with the substrate band 1312). Multiple substrate extensions 1314 may be configured to at least partially overlap. In some embodiments, one or more substrate extensions 1314 may have one or more contact points along the frame 1310 to enable stretching and/or crimping of the frame 1310 and/or substrate extensions 1314. The one or more substrate extensions 1314 may be configured to be bent (e.g., may be at least partially composed of an elastic and/or flexible material) to allow the one or more substrate extensions 1314 to be extended in an axial and/or circumferential direction.

The one or more substrate extensions 1314 may have a generally non-linear structure and/or may be at least partially curved. In some embodiments, the one or more substrate extensions 1314 may be configured to extend across one or more cells 1320 of the frame 1310. For example, the one or more substrate extensions 1314 may be configured to extend over empty space between struts 1315 of the frame 1310. At points where the one or more substrate extensions 1314 pass over struts 1315 of the frame 1310, the one or more substrate extensions 1314 may be configured to contact and/or attach to the frame 1310.

In some embodiments, a curvature of a substrate extension 1314 may advantageously allow the substrate extension 1314 to extend and/or otherwise mold in response to crimping of the valve 1300. For example, when the valve 1300 is crimped, a curvature of a substrate extension 1314 may be reduced and the substrate extension 1314 may form a more linear shape. The substrate extension 1314 may be composed of a generally flexible and/or elastic material. In some embodiments, the substrate extension 1314 may be configured to naturally return to an original curvature after the crimping process (e.g., when the valve 1300 is expanded from a crimped orientation).

The valve 1300 may be configured to include at least a first sensor 1305a coupled to the substrate band 1312 and/or situated at least partially within a first post 1340a extending from an end point at a first end portion 1350 of the valve 1300. The valve 1300 may additionally include a second sensor 1305b (e.g., situated within a second post 1340b and/or coupled to a substrate extension 1314), a third sensor 1305c (e.g., situated within a post 1340 and/or coupled to a substrate extension 1314), a fourth sensor 1305d (e.g., situated within a third post 1340c and/or coupled to a substrate extension 1314), and/or a fifth sensor 1305e (e.g., situated within a post and/or coupled to a substrate extension 1314).

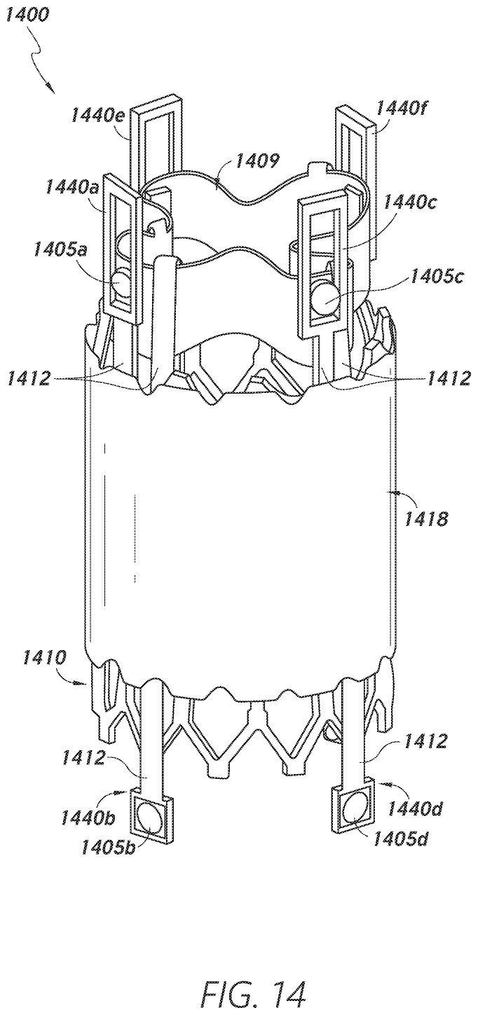

FIG. 14 illustrates a valve 1400 including a frame 1410 and one or more posts 1440 configured to allow one or more sensors 1405 to slide within the posts 1440 to adjust the positions of the one or more sensors 1405 with respect to the posts 1440 and/or frame 1410. For example, the one or more sensors 1405 may be configured to slide and/or move during crimping of the valve 1400. By allowing the sensors 1405 to slide with respect to the posts 1440, the positions of a first sensor 1405a at a first portion of the frame 1410 and a second sensor 1405b at a second portion of the frame 1410 may remain constant as the frame 1410 is crimped and/or compressed. In some embodiments, the valve 1400 may comprise a cover 1418 (e.g., composed of a polymer) configured to wrap at least partially around an outer surface of the frame 1410 and/or to enable growth of tissue for better integration of the valve 1400 with the surrounding tissue. In some embodiments, the valve 1400 may include one or more prosthetic leaflets 1409 configured to perform functions similar to valve leaflets.

The valve 1400 may include one or more substrates 1412 configured to extend one or more sensors 1405 to a first portion of the valve 1400 and/or to a second portion of the valve 1400. In some embodiments, one or more substrates 1412 may be configured to extend from the first portion to the second portion and/or from the second portion to the first portion.

The valve 1400 may have any number of sensors 1405 at a first portion (e.g., inflow portion) of the valve 1400 and any number of sensors 1405 at a second portion (e.g., outflow portion) of the valve 1400. Similarly, the valve 1400 may comprise any number of posts 1440 at the first portion of the valve 1400 and any number of posts 1440 at the second portion of the valve 1400. A first sensor 1405a may be situated at a first portion (e.g., an inflow portion) of the valve 1400 within a first post 1440a. A second sensor 1405b may be situated at a second portion (e.g., an outflow portion) of the valve 1400 within a second post 1440b and/or attached to a substrate 1412. The first sensor 1405a and second sensor 1405b together may be configured to measure a pressure differential across the valve 1400. Additional sensors 1405 may be configured to measure various parameters around a circumferential area of the valve 1400 to assist in detecting anomalous measurements. For example, the valve 1400 may further comprise a third sensor 1405c at a third post 1440c, a fourth sensor 1405d at a fourth post 1440d, and/or additional sensors 1405 at a fifth post 1440e and/or sixth post 1440f.

When the valve 1400 is crimped (e.g., during a delivery process into a patient's body), at least a portion of the valve 1400 may be configured to compress laterally (e.g., a diameter of an internal lumen of the valve 1400 may be reduced) and/or lengthen longitudinally (e.g., a distance between the first post 1440a and the second post 1440b may increase). In some embodiments, one or more sensors 1405 may be configured to slide within islets (i.e., receptors) of the posts 1440. For example, during crimping, a distance between the first sensor 1405a and the second sensor 1405b may remain unchanged even while the distance between the first post 1440a and the second post 1440b increases.

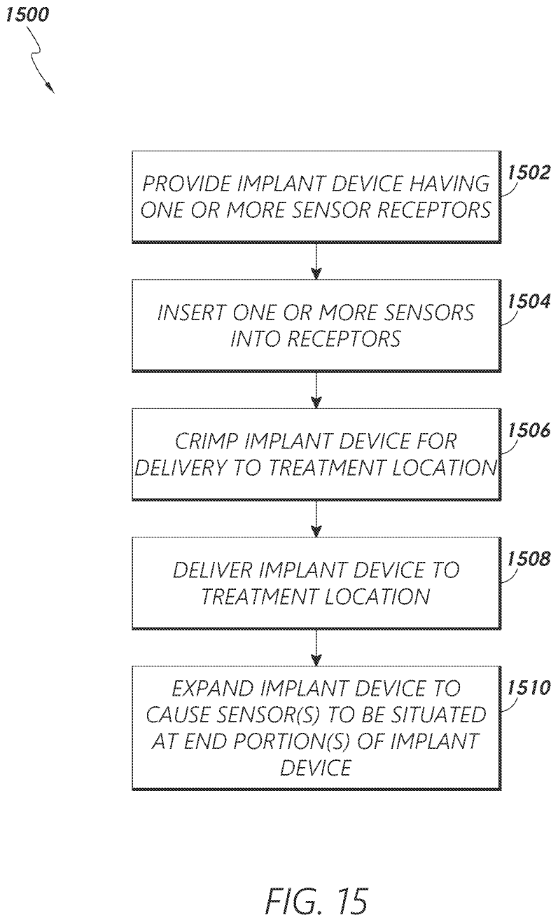

Disclosed herein are systems and devices which may be utilized in the monitoring of patients that have received implant devices, such as cardiac valve implant devices as disclosed herein. FIG. 15 is a flow diagram illustrating a process 1500 for monitoring a postoperative implant device and/or patient associated therewith. The process 1500 may be implemented at least in part by one or more of the entities or components of the systems shown in FIGS. 3 and 4 and described above. In some embodiments, the process 1500, or portions thereof, may be implemented by a physician or healthcare provider, or other user/entity.

The process 1500 involves, at block 1502, providing an implant device, such as a heart valve implant device, having one or more receptors for one or more sensors. In some embodiments, a receptor may include a post as described herein extending from a frame of a prosthetic valve. Receptors may additionally or alternatively include substrate bands and/or substrate extensions attached to a frame of a prosthetic valve. For example, one or more sensors may be attached to a substrate band forming a circular band around an interior or exterior of a cylindrical frame and/or one or more sensors may be attached to one or more substrate extensions extending from a substrate band. In some embodiments, receptors may be added to an existing implant device. One or more receptors may be situated at a first end portion of the implant device (e.g., an inflow portion) and/or one or more additional receptors may be situated at a second end portion of the implant device (e.g., an outflow portion).

At block 1504, the process 1500 involves inserting and/or attaching one or more sensors into/to the one or more receptors. For example, one or more sensors may be placed within posts extending from the implant device. In some embodiments, one or more sensors may be composed of a polymer and/or similar material and/or may have a generally soft structure. For example, one or more sensors may be configured to be molded and/or conformed to fit into variously-sized receptors, including posts as described herein.

At block 1506, the process 1500 involves crimping the implant device for delivery to a treatment location within a patient's body (e.g., the heart). Crimping may involve reducing a diameter of the implant device (e.g., reducing a diameter of an internal lumen of the implant device) and/or increasing a length of the implant device (e.g., increasing a distance between a first end portion of the implant device (e.g., an inflow portion) and a second end portion of the implant device (e.g., an outflow portion). Thus, crimping may increase a distance between a first receptor and/or a first sensor at the first receptor and a second receptor and/or a second sensor at the second receptor. In some embodiments, one or more sensors may be configured to slide within receptors during the crimping process. For example, a receptor may comprise a slidable post that may allow a sensor within the receptor to freely slide with respect to the receptor. In some embodiments, as the position of the receptor changes in response to crimping, a sensor within the receptor may maintain its position by sliding within the receptor. The crimped implant device may be placed within a catheter and/or other delivery device.

At block 1508, the process 1500 involves delivering the implant device to a desired treatment location. For example, the implant device may be delivered to an aortic valve of a patient. At block 1510, the process involves expanding the implant device to its original configuration prior to crimping. In some cases, removing the implant device from a catheter and/or other delivery device may cause expansion of the implant device. When the implant device reaches and/or returns to the expanded configuration, the one or more sensors may be situated at one or more end portions of the implant device. For example, a first sensor may be positioned at a first end portion and a second sensor may be positioned at a second end portion.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A prosthetic valve comprising:
a frame assembly having a first opening, a second opening, and an outer surface between the first opening and the second opening;
a sensor device positioned adjacent the first opening and configured to detect a pressure adjacent the first opening; and
one or more coils electrically coupled to the sensor device and positioned on the outer surface of the frame assembly, wherein the one or more coils and the sensor device form a circuit configured to transmit a signal indicative of the pressure adjacent the first opening.

2. The prosthetic valve of claim 1, wherein the one or more coils are attached to the outer surface of the frame assembly between the first opening and the second opening.

3. The prosthetic valve of claim 2, wherein the one or more coils comprise a shape that conforms to a shape of a portion of the frame assembly.

4. The prosthetic valve of claim 1, wherein the frame assembly comprises a network of struts forming cells, wherein the struts form end points at the first opening and the second opening, and wherein the one or more coils are secured to portions of the struts between the end points of the struts.

5. The prosthetic valve of claim 4, wherein the one or more coils comprise a shape that conforms to a shape of a portion of the struts of the frame assembly.

6. The prosthetic valve of claim 1, wherein the frame assembly is configured to be expanded and crimped, and wherein the one or more coils are attached to the outer surface of the frame assembly.

7. The prosthetic valve of claim 6, wherein the one or more coils are configured to (i) have an original form when the frame assembly is expanded, (ii) collapse when the frame assembly is crimped, and (iii) return substantially to the original form when the frame assembly is expanded after being crimped.

8. The prosthetic valve of claim 1, comprising a sheath at least partially covering the one or more coils, wherein the sheath is configured to at least one of provide electrical, thermal, and/or physical isolation between the one or more coils and the frame assembly.

9. The prosthetic valve of claim 1, comprising a sheath covering all or a substantial portion of the one or more coils and electrically isolating the one or more coils.

10. The prosthetic valve of claim 9, wherein the sheath comprises a biocompatible material.

11. The prosthetic valve of claim 1, wherein the circuit is a passive circuit.

12. The prosthetic valve of claim 1, wherein the sensor device comprises a capacitive element configured to detect a pressure, the one or more coils comprise an inductive element, and wherein the capacitive element and the one or more coils form an inductive-capacitive circuit.

13. The prosthetic valve of claim 12, wherein the inductive-capacitive circuit transmits a resonant frequency indicative of the pressure adjacent the first opening.

14. The prosthetic valve of claim 1, wherein the sensor device is a first sensor device, the one or more coils are one or more first coils, the circuit is a first circuit, the signal is a first signal, and wherein the prosthetic valve further comprises:
a second sensor device positioned at the second opening and configured to detect a pressure adjacent to the second opening; and
one or more second coils positioned on the outer surface of the frame assembly, wherein the one or more second coils and the second sensor device form a second circuit configured to transmit a second signal indicative of the pressure adjacent the second opening.

15. The prosthetic valve of claim 14, wherein the one or more first coils and the one or more second coils are configured to receive power for the first circuit and the second circuit, respectively, from a radio frequency (RF) field generated by a device external a body in which the prosthetic valve is deployed.

16. The prosthetic valve of claim 15, wherein:
the first circuit is configured to (i) generate, in response to receiving the power, the first signal indicative of the pressure adjacent the first opening and (ii) transmit the first signal via the one or more first coils; and
the second circuit is configured to (i) generate, in response to receiving the power, the second signal indicative of the pressure adjacent the second opening and (ii) transmit the second signal via the one or more second coils.

17. The prosthetic valve of claim 14, further comprising a third sensor device adjacent the first opening of the frame assembly.

18. The prosthetic valve of claim 1, wherein the sensor device is positioned on the frame assembly at a position so as to minimize restrictions on blood through the prosthetic valve.

19. The prosthetic valve of claim 1, wherein the frame assembly is configured to support a first post extending from the first opening of the frame assembly.

20. The prosthetic valve of claim 19, wherein the sensor device is situated at the first post.

21. The prosthetic valve of claim 20, wherein the sensor device is configured to slide within the first post.

22. The prosthetic valve of claim 1, further comprising a substrate band, wherein the sensor device is coupled to the substrate band.

23. The prosthetic valve of claim 22, further comprising a first substrate extension extending axially from the substrate band and in a direction toward the second opening of the frame assembly.

24. The prosthetic valve of claim 23, wherein a second sensor device is coupled to the first substrate extension adjacent the second opening of the frame assembly.

25. The prosthetic valve of claim 23, wherein the first substrate extension comprises a non-linear structure.

26. The prosthetic valve of claim 23, wherein the first substrate extension extends diagonally from the substrate band to the second opening of the frame assembly.

27. The prosthetic valve of claim 1, wherein the sensor device is composed of a polymer material.
